(19) United States
(12) Patent Application Publication
(10) Pub. No.: US 2008/0249362 A1
(43) Pub. Date: Oct. 9, 2008

(54) Endoscope System with a Disposal Sheath

(76) Inventors: Kerang Jiang, Shenyang (CN); Shoumei Jiang, Shenyang (CN)

Correspondence Address: JACOBSON HOLMAN PLLC, 400 SEVENTH STREET N.W., SUITE 600, WASHINGTON, DC, 20004, US

(21) Appl. No.: 10/574,485

(22) PCT Filed: Aug. 18, 2005
(86) PCT No.: PCT/CN05/01281

§ 371 Date: Apr. 3, 2006

(30) Foreign Application Priority Data
Dec. 14, 2004 (CN) 200420055751.5

(51) Int. Cl. A61B 1/012

(52) U.S. Cl. 600/121; 600/133

(57) ABSTRACT

The purpose of this invention is absolute elimination of medical cross-infection by endoscopy. It offers an endoscope system with a disposal sheath. The disposal sheath via anterior end cap connects the capsule which covers the outside of the endoscope and the disposal channel which sheathe in the endoscope channel and the fluid-air channel, and all these parts are joined together in a whole body. After the disposal channel is passed through the endoscope channel, its posterior disposal channel can be blocked into the elastic sealing cap via the guide tube, the posterior disposal channel's orifice need to be heated and melted and cut up simultaneously and its cut edge is in shape of V-shape by using one kind of heat fusion forceps after the use and before the retracting from the endoscope channel. A fluid-air exit and a sucking channel adapter are set on the posterior endoscope, convenience to connect with the fluid-air channel and the three-way sealing cap, the fluid-air channel may be single cavity channel or double cavity channel or two single cavity channels join in one channel together at nearing jet channel site, the channel sizes may be cylinders, or flat sizes. Both the outer surface of the endoscope and the inner surface of the endoscope channel are protected from pollution by the disposal sheath, thus ensures patients to safely use the permanent endoscope.

This is a nationalization of PCT/CN2005/001281 filed 18 Aug. 2005 and published in Chinese.

### TECHNICAL FIELD

This invention relates to endoscope and more particularly to an endoscope system with a disposal sheath for effective isolation of an endoscope from contamination on endoscopy, it is an improvement on traditional medical endoscopes to solve disposal sterilization, belongs to medical instrument field, suited for clinical use in hospitals.

### BACKGROUND OF THE INVENTION

Medical endoscope is used for diagnostic and therapeutic purpose during Micro-invasive surgery due to its incomparable function, because the morpho-characters and excision of focus can be directly observed and performed on endoscopy. But the terrible problem is medical cross-infection in particular some virus infection, such as Hepatitis and AIDS, via blood or other body fluid by endoscopy. Therefore, disposable small medical instruments should be recommended. However endoscopes as an interfering instrument are quite high cost at present. People have not found any excellent way to effect and conveniently sterilize and prevent endoscopes from damage simultaneously. By now autoclaved method is recommended for some rigid, endoscopes' sterilization due to application of some optical glues which could bear high temperature and high pressure. But flexible endoscopes can't bear with autoclaved way, also disinfection solution can't thoroughgoing flow into varied channels to sterilize wholly. Since ninety decade last century, some foreign patents discussed the difficult issues thereinbefore. They generally set a disposal sheath on outside endoscope and transform endoscope body (this patent's applicant, JIANG, Kerang, also provides some patent applications numbered 03136141.2 and 02158217.3 et al ), and disposal channel is settled on outside of the endoscope channel. The U.S. Pat. No. 5,050,585, provides that of the disposal channel settled in inside of traditional endoscopes, and the anterior opening of disposal channel connected with the disposal sheath which covers on the endoscope to protect endoscope's outer surface and inner surface of opened channel, that is a good idea. Japanese Pentax company on this basis applied lots of patents for improvement later on. However, as to information from applications' publication, the cross-infection problem of medical endoscope in clinic application has yet not solved. In order to produce this kind of product, it will have yet a quite long way to go. More details are also needed to improve, such as fluid-air channel's position in endoscope system and other located connecting structure and correctly treat the disposal channel after use etc. All of such problems must be solved by new way.

### SUMMARY OF THE INVENTION

This invention provides a safe matching assembly system with an endoscope disposal sheath, to solve medical cross-infection thoroughly. That is to put forward newly program, improve lots of details, and reach clinical practice.

This technological program of this invention is as follows:

For protecting an improved permanent endoscope, the innovative uses a disposal sheath to ensure the endoscope not contaminant in clinic application. The said disposal sheath consists of a end cap, a capsule, a disposal channel, a soft double cavity fluid-air channel, a jet channel, a locking ring. Said end cap connects the disposal capsule which located in outside endoscope and the disposal channel which located in the endoscope channel and the soft double cavity fluid-air channel, thus all together connect with and join in a whole body, then to protect the endoscope's outer surface and channel's inner surface as well. The end cap is made from some elastic or partial elastic transparent materials, its inner end face's size fits in the end face's size of anterior endoscope. The end cap's inner face matches the endoscope body. A jet channel orifice is coaxially set at the anterior end cap to fix the jet channel. The space between the ditch and the channel need to tight glue with certain adhesive. A fixing seat or a fixing orifice of the disposal channel is set at the endoscope's operation orifice's position in the end surface of the end cap for convenient connecting with the disposal channel. The outer surrounding of posterior end cap is sheathed in the anterior end of the capsule. The capsule is made from elastic flexible thin wall nonpoisonous, non-immune biocompatible and lubricant or lubricated, medical material. The capsule covers the outside of endoscope. The posterior capsule connects a locking ring. While pull to lengthen, the elastic capsule can be locked by the locking ring to ensure the anterior inner face of the end cap constantly and tight close to anterior end face of the endoscope. Since no space between the capsule end cap end anterior tip of the endoscope, reflected complicated light formed by lighting ray at observation system can be decreased. The upper and lower handles of the locking ring have corresponding oblique teeth to tighten or loose the posterior capsule. The fluid-air channel is connected to posterior end of the jet channel, and located in between the endoscope and the capsule. the fluid-air channel can be a single cavity or double cavities, or the structure of two single cavity channels together join in one cavity channel at nearing jet channel site, its channel's size can be cylinder, or flat orifice, said the structure of single cavity channels, may be two flat channels which is formed via heat pressure, and parallel together with each other thin wall channels. The flat channels is not easy to be bent and to block the flowing fluid-air also can be inflated and expanded into two cylinder channel cavities while flowing fluid-air flow into them, said the structure of double cavity fluid-air channel. Inside of the anterior part of it which sheathes on the jet channel has not any septum, but inside the later part of the double cavity channel has a septum, and at the back of the jet channel anterior septum has a round head which fits in the inner wall of the channel cavity, and there is a section of incision, respectively at situation of the septum's two sides, separated from the channel wall in order to make the septum swing towards any side of channel cavity to block the channel which the flowing fluid needs not flow into; The posterior soft double cavity channel(including two channels) is sheathed on two rigid connecting tubes that is has two parallel channels which are welded together, the posterior orifice of two rigid connecting tube are sheathed in two adapters of the two soft channels connecting with fluid and air channels respectively. The jet of the jet channel which is connected with the anterior double cavity channel, sprays out the fluid or air and direct towards the outer surface of the end cap and fits in the object lens situated inside of the end cap. The disposal channel is made of flexible, durable, hard to bent, bear negative pressure and electric arc-resistant medical materials, its anterior tip connects with the end cap. Its connecting method includes to directly sheathe and glue the disposal channel on the end cap by set a channel seat or to sheathe by a channel seat which can bear high temperature and be electric arc-resistant, glued in fixing orifice of the end cap.

For the use of matching the disposal sheath, the endoscope body needs to be improved as follows:

\textcircled{1} The original anterior endoscope's fluid-air channel and jet channel are removed, and placed into the position outside of the endoscope, only a fluid exit and a air exit are set on posterior endoscope below the handle. A sucking channel adapter is also set near the fluid-air exits mentioned above.

\textcircled{2} The diameter of operation channel(endoscope channel) is enlarged properly, for being convenient to insert disposal channel into it.

In addition, this invention also provides a kind of three-way sealing cap, a guide tube, a sucking tube and two kinds of heat fusion forceps which can shear disposal channel to V-shape cut edge. All these methods can isolate the endoscope channel from the contaminated disposal channel.

The three-way sealing cap is made from elastic material, the straight cavity is used for fixing and sealing the posterior end of the disposal channel and used as entrance or exit of instrument, its anterior end can be fixed on the exit of the endoscope channel, and on one side of the straight cavity set a sucking channel adapter used as a sucking way connecting the sucking tube via the disposal channel. Three elastic sealing orifices are set in the straight cavity. The inner diameter of three elastic sealing orifices is less than the outer diameter of the disposal channel. In order to solve the difficulty of inserting the disposal channel, the invention provides a kind of guide tube. The inner diameter of the guide tube is more than the outer diameter of the disposal channel. The outer diameter of the guide tube is more than the inner diameter of three sealing orifices of the three-way sealing cap. Due to the guide tube is made of rigid thin wall of tube, it can be, in advance, inserted into three-way sealing cap as to put the disposal channel into it with convenience. After the three-way sealing cap was fixed on, the guide tube is pulled out, then the disposal channel is kept in the three-way sealing cap. In order to let the disposal channel is correctly put in the exit of the second elastic sealing orifice, a tube core is set in the guide tube and the over-length part which is heightened than the sucking channel orifice can be pushed to the above mentioned application position by the lower end of the tube core. The inserting deepness of the guide tube core depends on the distance between the handle limiting face and orientation pin. The orientation pin is pushed to in front of the third elastic sealing orifice by the posterior end of the guide tube's long orifice, and to be blocked, during the process of pulling the guide tube out although the lower end of the guide tube leave away from the first elastic sealing orifice, the disposal channel, instantly is held by the first elastic sealing orifice, cannot turn over to raise till the guide tube bring the tube core continuous pulled out so that the disposal channel again be blocked in the second elastic sealing orifice of the three-way sealing cap and be then sealed and be keep-held, till the guide tube is pulled out wholly.

While the disposal sheath and the endoscope being in use, the first thing is to insert the disposal channel into the endoscope channel, later the other part of the disposal sheath is sheathed into it respectively. For solving the difficulty of inserting the disposal channel, to begin with, it is about to put an endoscope instrument, such as biopsy forceps into the endoscope channel, besides, to cover the instrument's head part by using the disposal channel and stretch out the endoscope head. While the instrument is in fixing position, not to remove, the disposal channel can be smoothly passed into the endoscope channel along a space between the endoscope instrument and endoscope channel. Then outside of endoscope can also be covered on by another part of the disposal sheath. After that, to pull upwards the locking ring to nearing handle position on endoscope body, then to firmly lock it. Thus the capsule's elastic thin membrane makes the end cap's inner end and anterior endoscope end's surface tight close with each other. There is clear vision field so far. At that time posterior end of the disposal channel already extends to exterior of the endoscope channel exit, the anterior end orifice of the guide tube which was inserted in the anterior three-way sealing cap sheathes on outside of posterior end orifice of the disposal channel, sheathes forwards and the three-way sealing cap is fixed on the endoscope channel exit site and the sucking channel connecting with the three-way sealing cap is connected to the sucking channel adapter, it can be used in clinic application after pulling out the guide tube when finishing this process , and two channels (fluid channel and air channel) are connected to two exit (fluid exit and air exit) of the endoscope respectively.

After the clinic use, the three-way sealing cap and the sucking channel need to pull out(the three-way sealing cap and the sucking channel are made of high temperature resistant material so that they can be reused after cleaning and disinfecting, of course also can be disposal), then the disposal sheath need to pull out. Due to the disposal channel orifice has been contaminative, the outlet of the disposal channel must be well blocked. After be blocked and then extracted, this can avoid the endoscope channel from pollution. The endoscope channel can be protected and prevented from becoming re-polluted source in the later use. So this invention provides two kinds of heat fusion forceps for cutting the outlet of the disposal channel and fusing certain shape of the disposal channel at the position nearing the posterior outlet of the disposal channel. This shall ensure sheared the disposal channel under certain sterilization temperature when exiting. While fused and melted simultaneous the disposal channel to be cut out. The cut edge's size also need to meet certain requirement, that is while the sheared outlet of the disposal channel passed the endoscope channel, the cut-edge's shape becomes a V-shape, the disposal channel's shape still is a full cylindrical not to be squeezed and opened, the cut edge's shape needs to become a V-shape. Thus the outlet of the disposal channel is in favor of not reopening. This invention provides two kinds of V-shape cut edge which can satisfy the goal. Structure of the one of the heat fusion forceps is to set a piece of electric resistance thread in axial direction with V-shape at the forceps exit plane (the V-shape electric resistance thread also can be installed in contradirectional position), to set a flexible insulation board at an opposite plane's piece which can freely swing. The two planes are parallel with each other and can be freely closed. The second kind of heat fusion forceps has two pieces structures, piece-upper and piece-lower with a same degree, formed positive and negative V-shapes forceps exit. One piece can be moved radially and swing relatively for parallel pressurization to the channel exit. After heat the electric thread, the sheared outlet of the disposal channel can be changed into V-shape in radial direction as the first kind of heat fusion forceps also can achieve the same effectiveness.

Compare to present technology, this invention has following advantages:

\textcircled{1} Both US 2004/0077927A1 and Japanese 2004-129813 patents offered a tight turn method to shorten the space between capsule end cap and anterior end of endoscope in order to decrease reflected complicated light. But turning space of the endoscope channel certainly increased, so that it occupies precious radial space in endoscope. In this invention we use elasticity of the sheath to made elastic tight locking the protrusion platform of end cap, and saved the occupying space.

\textcircled{2} Japanese patent application, 2003-153848, provides a method to block the disposal channel before removing from the endoscope channel after use. That method uses a pair positive-negative of C-shape R metal block to heat and melt the disposal channel and then cut the disposal channel by using a knife. Its inconvenience is that the positive-negative of C-shape R Ratio changes a lot depending on the thickness of the heated channel wall. This invention provides two kinds of V-shape heat fusion forceps, which are easy and convenient during the operation, and the V-shape's angle does not relate to channel wall's thickness.

\textcircled{3} Compared with other aspects of present technology, the invention also provides more advantages such as the three-way sealing cap's structure, a special guide tube and traction forceps different kind of fluid-air channel and its septum, and the anterior part of the septum can swing to avoid the back-flow function etc. It also has unique advantage and special points to arouse elastic sealing cap smoothly fixing in the disposal channel.

### BRIEF DESCRIPTION OF THE DRAWINGS

FIG. 1 is a whole body's appearance of this invention.

FIG. 2 shows the structure figure of a disposal sheath.

FIG. 3 shows the double cavity fluid-air channel.

FIG. 4 is the overlooking figure of the FIG. 3.

FIG. 5 is a fluid-air channel consists of two flat channels.

FIG. 6 is a head part's structure figure of the disposal sheath.

FIG. 7 shows another kind of head part's structure figure of disposal sheath.

FIG. 8 is a head part's (having thin membrane) structure figure of disposal sheath.

FIG. 9 shows is a locking ring.

FIG. 10 is a three-way sealing cap figure.

FIG. 11 is a guide tube figure.

FIG. 12 shows a guide tube inserting into three way sealing cap and a disposal channel for preparation to sheathe and shows endoscope channel outlet.

FIG. 13 shows a disposal channel is already sheathed into three way sealing cap and a disposal channel.

FIG. 14 shows preparation of pulling out the guide tube after the process of FIG. 13.

FIG. 15 shows a kind of V-shape heat fusion forceps.

FIG. 16 shows a section view of a V-shape heat fusion forceps at closing forceps' position as shown in FIG. 15.

FIG. 17 shows another example of the structure of V-shape head fusion forceps.

FIG. 18 shows two samples of a disposal channel's outlet have been cut off by a forceps as shown in FIG. 17.

FIG. 19 shows a structure figure of a traction forceps.

FIG. 20 is a magnified figure of head part of the traction forceps in FIG. 19.

In figures: 1 is an endoscope, 2 is an end cap, 3 is a disposal channel, 3.1 is a channel seat ,3.2 is a V-shape channel orifice, 3.3 is a other V-shape channel, 4 is a fluid-air channel, 4.1 is a septum cavity, 4.2 is septum of round head, 4.3 is double channels adapters, 4.4 is connecting material of double channels adapters, 4.5 is rigid curved channel, 4.6 is single cavity channel, R1 is curve rate of channel cavity, R2 is curve rate of septum end head, 5 is a jet channel, 6 is a fluid-air channel adapter, 7 is a fluid-air exit, 8 is a capsule, 9 is a locking ring, 10 is an endoscope channel, 11 is an endoscope channel exit, 12 is a three-way sealing cap, 12.1 is the first elastic sealing orifice, 12.2 is the second elastic sealing orifice, 12.3 is the third elastic sealing orifice, 12.4 is a lower adapter, 12.5 is a sucking channel adapter, 12.6 is an upper orifice, 13 is a guide tube, 13.1 is a guide tube core, 13.2 is a handle, 13.3 is an orientation pin, 13.4 is a long orifice, 13.5 is a lower orifice of guide tube, 13.6 is an end face of handle, 13.7 is a lower end of tube core, 13.8 is a tube body, 14 is a kind of heat fusion forceps, 14.1 is a lower forceps orifice, 14.2 is an electric resistance thread, 14.3 is an upper forceps orifice, 14.4 is a wire, 15 is an another kind of heat fusion forceps, 15.1 active board 16 is a sucking channel adapter 17 is A sucking channel.

### DESCRIPTION OF THE INVENTION IN DETAIL

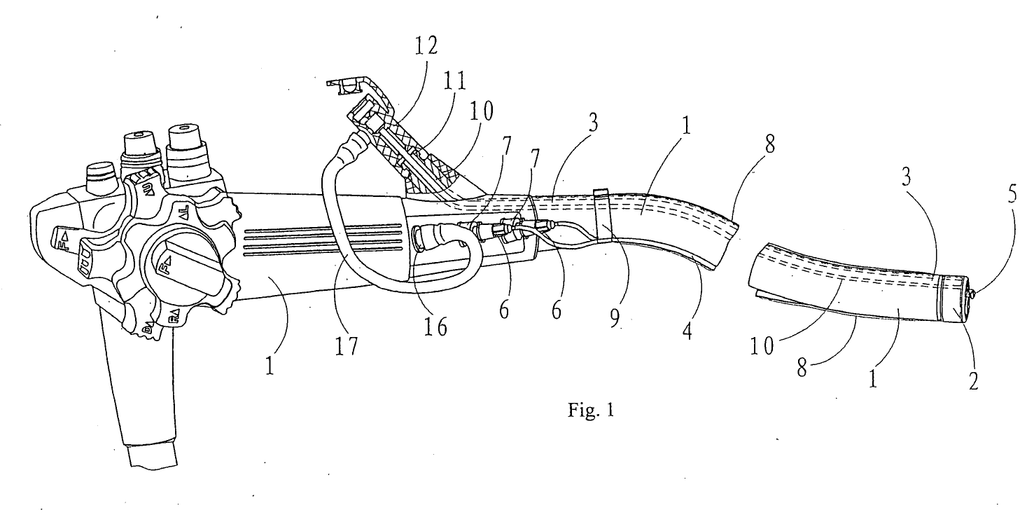

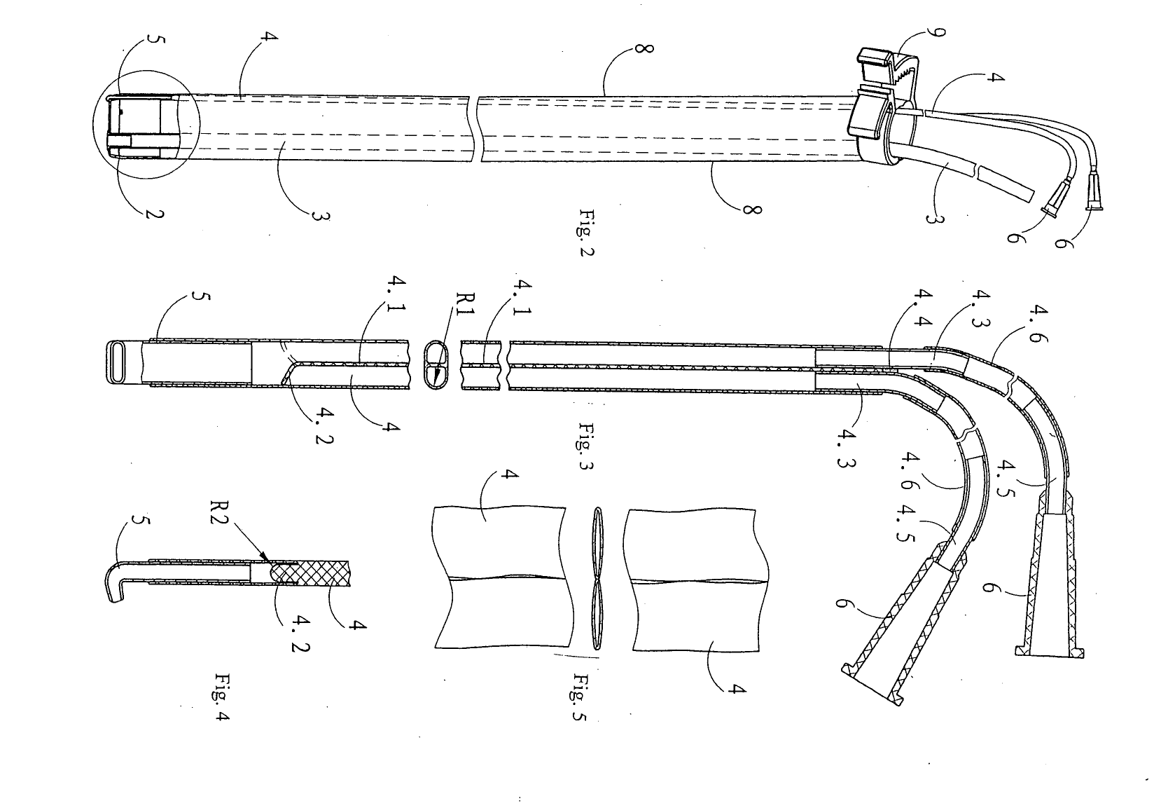

This invention consists of two parts, an improved permanent endoscope and a disposal sheath, altogether formed an endoscope system with the disposal sheath (see FIG. 1). It offers the permanent endoscope, which can be used for many times, to be protected by the disposal sheath from pollution. The disposal sheath (see FIG. 2) not only has the capsule 8 and the end cap 2, to protect the endoscope1outer-surface, but also has a disposal channel 3 placed into the endoscope channel 10 to protect the endoscope channel's inner-surface. The capsule and the disposal channel are connected through the end cap 2. And they are joined in a whole body, therefore, the endoscope of both inside and outside are protected in all.

The detail of above-mentioned endoscope system with a disposal sheath is as follows:

At first, to reform the traditional endoscope below:

In the anterior endoscopel, there is not any fluid-air channel or jet channel. The fluid-air channel and the jet channel are removed to the outside of the endoscope position between the endoscope 1 and the capsule 8. The jet channel is fixed in the jet channel ditch which located in one side of the end cap, the fluid-air exit 7 and the sucking channel adapter 16 are set on the posterior endoscope. The fluid-air exit 7 and the sucking channel adapter 16 connect the fluid-air channel adapter 6 and the sucking channel 17 respectively. The sucking channel adapter 12.5 of the three-way sealing cap 12 sheathed with another end of the sucking channel 17.

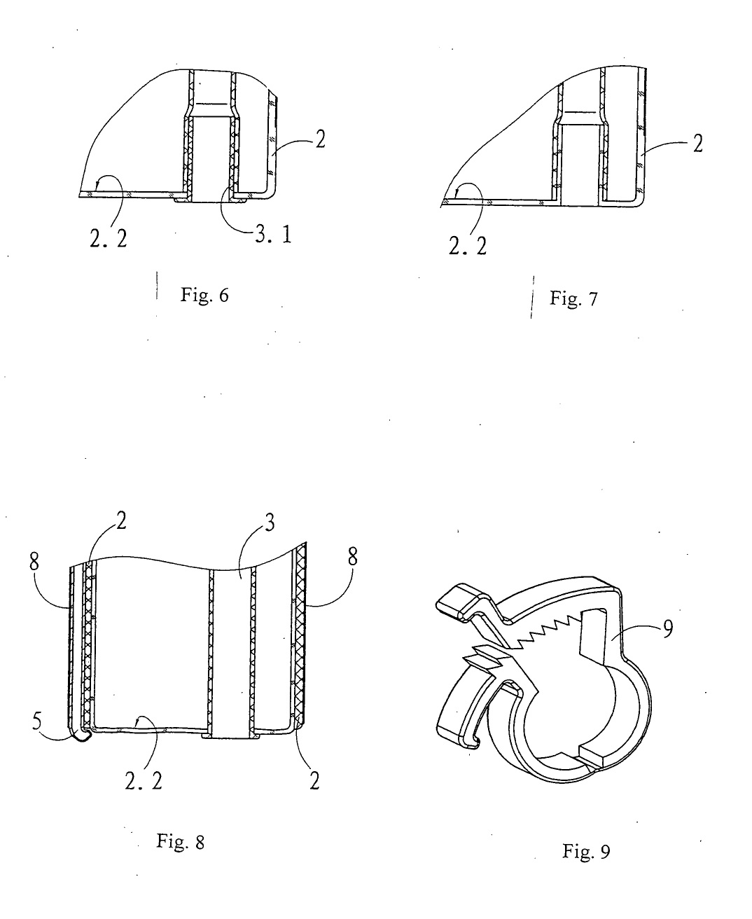

On the other aspect, the disposal sheath consists of the end cap 2, the capsule 8, the disposal channel 3, the locking ring 9, the fluid-air channel 4, the jet channel 5, the fluid-air channel adapter 6, the three-way sealing cap 12, the sucking channel 17 and guide tube 13 etc . The invention also provides the traction forceps and two kinds of heat fusion forceps 14 and 15 for simultaneous melting and cutting the disposal channel 3 after the use. the end cap 2 is made of transparent or partial flexible transparent material. To decrease the complication light, out of illumination outgoing light and reflect into observation system, this invention provides two kinds of program: due to conventional endoscopes' anterior end surface are not flat, and have different size, the invention offers the end cap as shown in FIG. 8. The end surface of the end cap is made of elastic, flexible, thin membrane, so that it can close to the illumination out-going light and the observation system while it is pulled and tightened and the complication light reflected between them can be decreased. For this reason, the invention provides special endoscope-its anterior end surface is quite flat. As shown in FIG. 6 and FIG. 7 a kind of end cap, improved by this invention, processes its flat anterior inner surface. This makes closely contact possible between anterior end surface of endoscope and inner end surface of the end cap 2 which can decrease complication light as well. The posterior end of the jet channel 5 is connected with the fluid-air channel. The jet orifice of the jet channel 5 directs towards, outer end surface of the end cap relative to the object lens' position, and buckle up in the through ditch 2.3, its posterior end sheathes the fluid-air channel 4 the exit of the jet channel 5 directs towards the outer surface of the end cap relative to the object lens site. Anterior end cap, similar with anterior end of the endoscope, at the position of endoscope channel, opening the end surface of end cap 2 connects with the disposal channel 3. there are three connection ways: 1) as shown in FIG. 6, adopting the channel seat 3.1 to connect the end cap 2 with adhesive; 2) as shown in FIG. 7, the channel seat 3.1 and the end cap 2 formed a whole body; 3) as shown in FIG. 8, without any channel seat, the tip of the disposal channel turned over outwards. The capsule tight covers the outside of end cap 2. The parts that the end cap 2 connected with including the capsule 8, the jet channel 5, the disposal channel seat 3.1, the disposal channel 3, all applied with medical adhesive.

The capsule 8 is made of lacto rubber thin membrane material, or PVC thin membrane tube or polyaminoesters, or other high molecular material etc. Anterior end of the capsule 8 adhesively connects with the outside of posterior end cap 2 or majority of the outside of end cap 2. The length and diameter of the capsule 8 matches the length and diameter of the endoscope body, its posterior end connects to the locking ring 9. All of those connections are adhered with adhesive.

The disposal channel 3 is made from sturdy, elastic, high temperature resistance, electric arc resistance and bear negative pressure material, its length and diameter matches the length and diameter of the endoscope channel. Its anterior end is adhered on the end surface of the end cap 2, sites in the endoscope channel. In order to solve the inserting difficulty of the disposal channel 3, the operation can be: firstly to insert an instrument such as biopsy forceps into the endoscope channel 10, let the instrument's head surpass anterior end of endoscope channel, then outside of the instrument sheathes in the disposal channel, they are inserted into the endoscope channel 10 altogether. As shown in FIG. 19 and FIG. 20, a traction forceps can be used, if necessary, to pull the disposal channel 3 passing through the endoscope channel 10. The traction forceps has two pieces of forceps piece, one piece is a cylinder and other piece is a half bullet size for contacting most area of channel wall. The lock of the forceps handle has some locking teeth While the disposal channel 3 passes through the endoscope channel, the capsule 8 and the end cap 2 are pulled by the locking ring 9 and heightened. Two fluid-air channels adapters 6 sheathe on the fluid-air exit 7 of the endoscope. The fluid-air channel can be single cavity or double cavity channel or the structure of two single cavity channels are joined together in one channel at nearing jet channel site , the channels shape may be cylinders, or flat shape as shown in FIG. 5 the fluid-air channel consisted of two single cavity channels ,may be consisted of two flat channels, which is formed via heat pressure ,and parallel together with each other. The advantage of the flat channel is can not bent and can not be blocked by the flowing fluid-air, in addition, the closing channel which is not entered into by the flowing fluid-air only occupies less space. as shown in FIG. 3, 4) said of the structure of two fluid-air channel 4, it has not any septum inside the sheathed part with the jet channel 5, but inside the later part of the double cavity channel has a septum cavity 4.1 and on the anterior septum part of the double cavity channel at the back of the jet channel swinging set a septum of round head 4.2, its curve rate of double cavity septum head R2 fits the curve rate of the channel cavity. There are two incisions separating from the cavity wall, located at two sides of the certain section of the septum of round head 4.2, the septum section of round head 4.2 can be swung towards the left or right, to block the one of two cavities of the channel which flowing fluid needs not to flow into, prevent injecting fluid or air from back flowing into this one cavity. The posterior end of the soft double cavity channel sheathes on the rigid double channels adapters 4.3, between the double channels there is a connecting material (in this application example is a welding pad piece), the posterior end of double channels adapters sheathe in two single cavity channels 4.6, Which again sheath on two rigid curve channels 4.5 connecting the fluid-air channels adapters in order to convenience connect with two fluid-air exits of the endoscope.

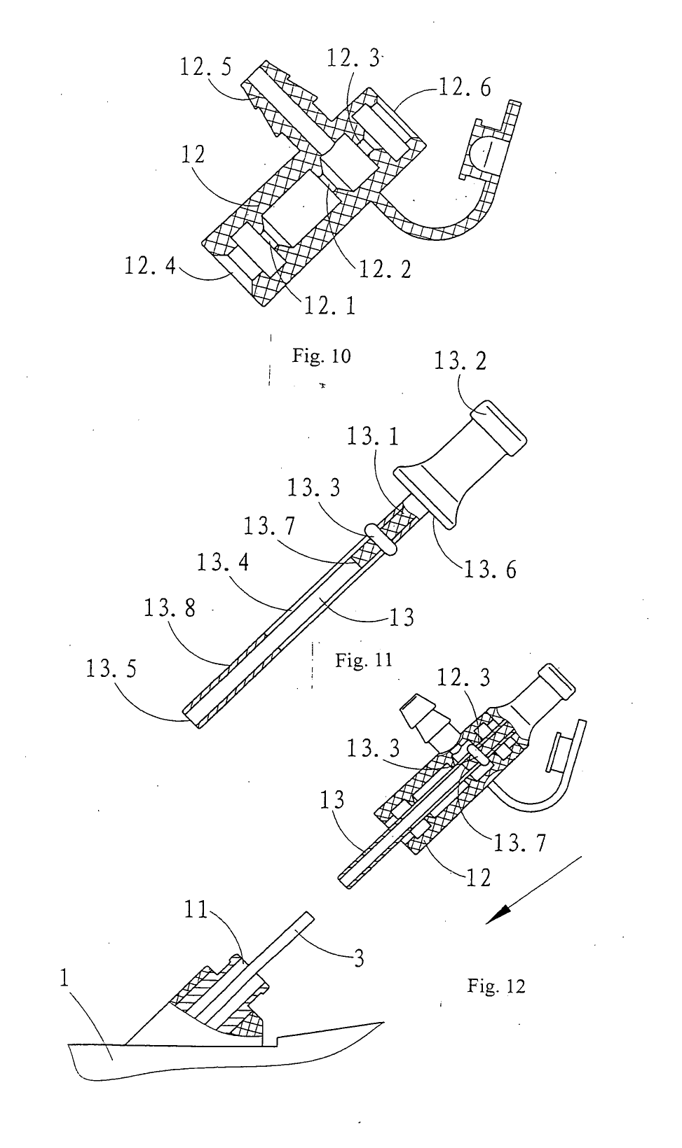

At that time, the part of the posterior end of disposal channel 3, which passed through the posterior end of the endoscope 11, needs to sheathe in a three-way sealing cap 12 as shown in FIG. 10. The straight cavity of the three-way sealing cap 12 has upper adapter 12.6 and lower adapter 12.4. The first elastic sealing orifices 12.1, the second elastic sealing orifices 12.2, and the third elastic sealing orifices 12.3 are also set inside the straight cavity of the three-way sealing cap 12. At one side of the three-way sealing cap 12, the sucking channel adapter 12.5 is set for sheathing the sucking channel 17. The sucking channel 21 connects with sucking channel adapter 16. On posterior endoscope, the sucking channel adapter 20 connects with some relative channel or handy valve inside the endoscope. To suck the anterior disposal channel's fluid into fluid reserve by handy valve when needed.

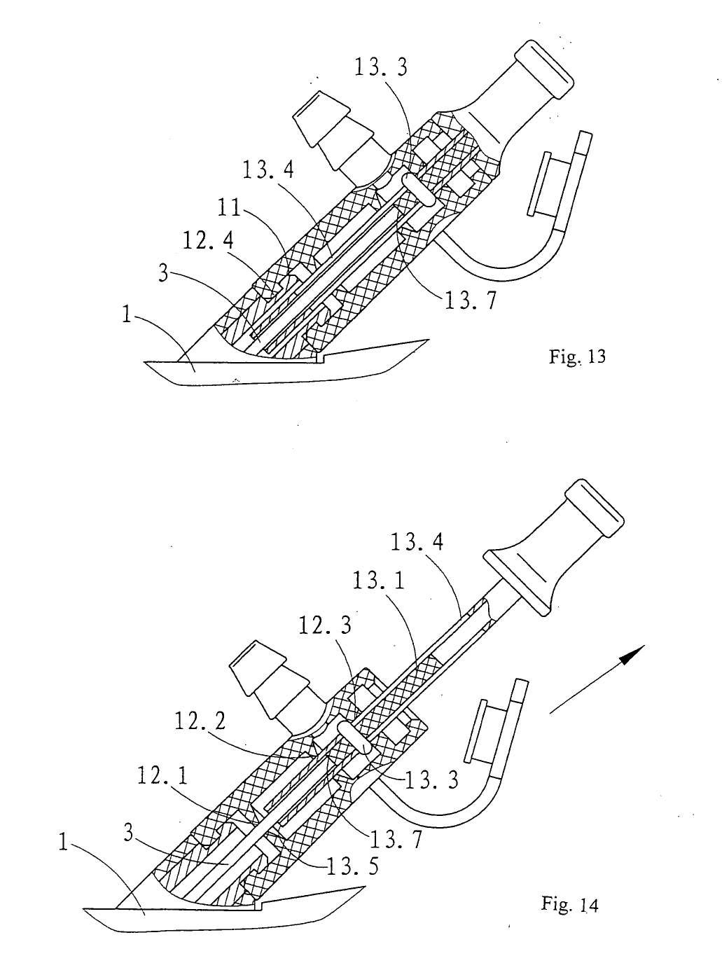

Owing to the inner diameter of the first to third elastic sealing orifices 12.1, 12.2, 12.3 of the three-way sealing cap 12 is less than the outer diameter of the disposal channel 3, for solve the inserting difficulty of the disposal channel 3, this invention also provides the guide tube 13 as shown in FIG. 11. The guide tube 13 consists of a tube body 13.8 with a handle 13.2, a guide tube core 13.1, a long orifice 13.4, a orientation pin 13.3. The tube body 13.8 is made of thin wall tube. A certain length, symmetrical long orifice 13.4 is set at surface of its one side, to let the orientation pin 13.3 across the tube core 17.1 conveniently and slide in it. While match the guide tube with the three-way sealing cap(as shown in the FIG. 1) the distance between the handle end face 13.6 and posterior end of long orifice equals to the distance between the upper orifice of the three-way sealing cap and lower end face of the third elastic sealing orifice. The long orifice's length is more than the distance between the lower end of the guide tube and the first elastic sealing orifice of the three-way sealing cap. While the three-way sealing cap 12 is prepared to sheathe in the disposal channel 3 and fix on endoscope channel exit, the followings are needed to be done: firstly to let the guide tube 13(as shown in FIG. 12) sheathe in the three-way sealing cap 12, then to aim the lower end orifice 13.5 of the guide tube 13 at and cover the disposal channel orifice. While the three-way sealing cap 12 and sheathes on the endoscope (as shown in FIG. 13) over length posterior end of disposal channel 3 is pressured to lower end of the sucking orifice by lower end of tube core 13.7. The orientation of the lower end of tube core 13.7 depends on the deepness decided by the orientation pin 13.3 and handle end face 13.6. As shown in FIG. 12, the process of pulling the guide tube 13 out, at the time that lower orifice of guide tube 13.5 leaves the first elastic sealing orifice 12.1, the lower end of tube core 13.7 is yet locked on the front of the third elastic sealing orifice 12.3 due to the orientation pin. After the disposal channel 3 is contracted and fixed in the first elastic sealing orifice 12.1, till the guide tube 13 and tube core 13.1 are pulled out by the handle 13.2, as shown in FIG. 14, the orientation pin 13.3 had retracted out of the third elastic sealing orifice 12.1, the lower end of tube core 13.7 no longer limit the disposal channel 3. The disposal channel is tight fixed in the first elastic sealing orifice, at that time again had tight fixed and sealed in the second elastic sealing orifice 12.2.

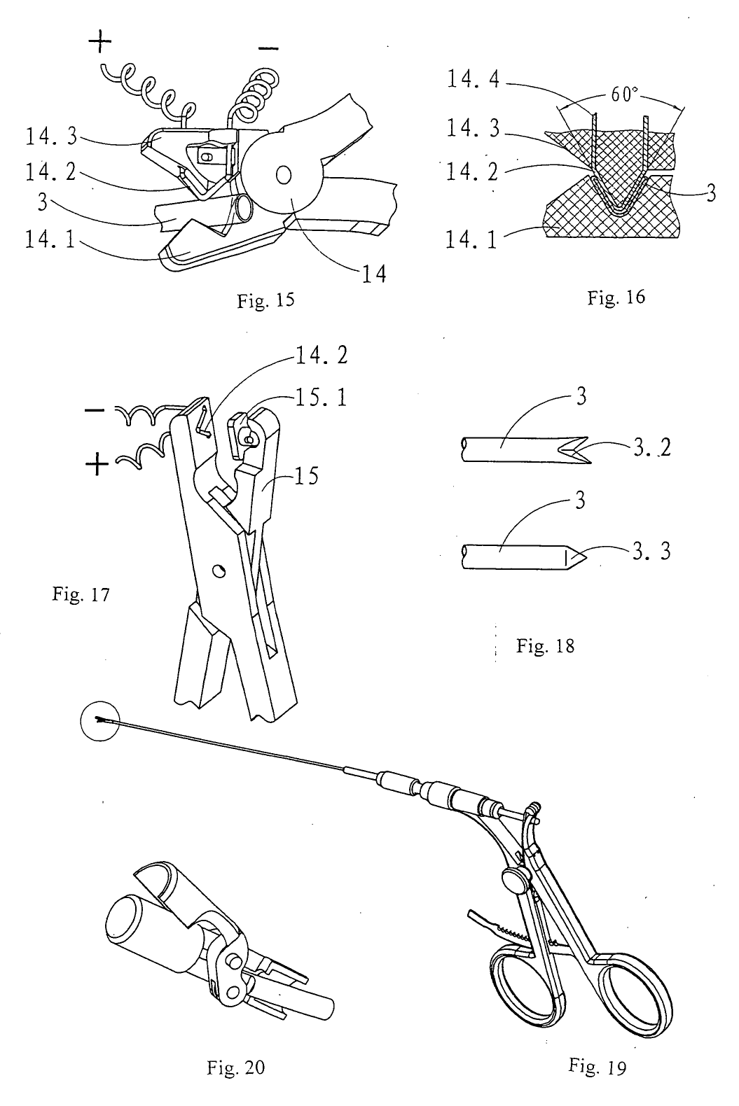

After clinic use, the disposal channel has contaminated all around, must be taken off treated as medical rubbish. Before the disposal channel 3 is taken out from the endoscope channel 10, the posterior end of channel orifice must be blocked, otherwise the endoscope channel 10 will be polluted. The blocking method must to ensure sterilizing the channel orifice and not reopen the channel orifice because of its bad size during the taking out process. Therefore, this invention provides two kinds of heat fusion forceps 14 and 15 (respectively as shown in FIG. 15 and FIG. 17). The disposal channel's orifice can be cut off and formed a V-shape by heating the electric resistance thread to one piece of two pieces of forceps exit. The cut-edge's temperature reaches sterilization condition. Said two forceps concrete structures are as follows: one is, as shown in FIG. 15, the kind of heat fusion forceps 14, its channel orifice forms V-shape in radial direction V-shape electric resistance thread 14.2 in radial direction is parallel set on one piece of forceps opening. A cut edge by the heat fusion forceps of the disposal channel 3 formed a radial V-shape section as shown in FIG. 16. The action of the forceps piece 14.3 makes the channel orifice balance and symmetric heating fusion; another heat fusion forceps 19 structured an axial direction V-shape of cut edge as shown in FIG. 17. A movable insulation active board 15.1, set on one forceps piece, can form symmetric heat fusion. Its cutting edge forms a V-shape as shown in FIG. 18. According to tests, the disposal channel 3 can basically be full cylindrical shape of channel for ensure after cutting off by this method, therefore, can be securely taken off from the endoscope channel 11 in favor of the blocked channel orifice not reopening. As shown in FIG. 18, the disposal channel 3 formed an axial direction V-shape cut edge of channel-other V-shape channel 3.3 after cutting off by the heat fusion forceps (FIG. 17). As shown in FIG. 17, the electric resistance thread 14.2 of the forceps orifice can be set and swung contradirectionally the same effeteness to the formed V-shape cut edge 3.2. Due to the three-way sealing cap 12 and the sucking channel 17 are made of materials which can bear high temperature, so that they can be reused after cleaning and disinfecting, of course also can be disposal. Since the sucking channel 13 did not contact with contamination at all, it can be cleaned by common method.

What is claimed:

1. An endoscope system with a disposal sheath, including an permanent endoscope which can be reused for many times and a disposal sheath; wherein the capsule covers the outside of the endoscope as a part of the disposal sheath, connects the disposal channel which is inserted into the endoscope channel, both the anterior ends of the capsule and the disposal channel are connected and joined in a whole body; characterized in that a fluid-air exit and a sucking channel adapter are set on posterior endoscope, sheathe and connect a fluid-air channel adapter and sucking channel adapter of the disposal sheath respectively, the fluid-air channel can be a single cavity or double cavities channels; the fluid-air channel is set on outside of the endoscope; and parallel with the endoscope; the capsule, as the main body of the disposal sheath, covers the outside of the endoscope joining the jet channel and the fluid-air channel via the end cap on the anterior end of capsule to form a whole body; the disposal sheath covers the outer surface of the endoscope body and the inner surface of the endoscope channel as well; the posterior capsule connects a locking ring; after passing through the endoscope channel, the disposal channel is positioned in the three-way sealing cap via the guide tube; after the use, the channel orifice of the disposal channel can be heated and melted and cut off by a heat fusion forceps, to form a blocked V-shape.

2. The endoscope system with a disposal sheath, according to the claim 1, characterized in that the end cap is made of transparent material or partial elastic transparent thin membrane, its inner end face's shape coincides with anterior end face of the endoscope body, the disposal channel connects in the endoscope channel at axial direction in anterior endoscope's site of the end cap, the end cap's surrounding is sheathed in anterior capsule.

3. The endoscope system with a disposal sheath, according to the claim 2, characterized in that said the connection between the disposal channel and the end cap is via a channel seat or a turn-over outwards' adapter which is produced on the disposal channel tip.

4. The endoscope system with a disposal sheath, according to the claim 1, characterized in that the capsule is made from elastic, flexible thin wall nonpoisonous, non-immune, biocompatible and lubricant or lubricated material, the anterior capsule covers the posterior end cap's surrounding, the posterior capsule connects the locking ring, the locking ring the upper and lower oblique teeth and the handle of the locking ring can make the capsule fix or loose the endoscope, and can be tight locked up or unlocked by handle.

5. The endoscope system with a disposal sheath, according to the claim 1, characterized in that the fluid-air channel can be made from double cavities channel, in its interior section which sheathe on the jet channel has no any septum, in order to convenient to sheathe the jet channel: Inside the later part of the double cavity channel has a septum, and the anterior septum at the back of the jet channel is a round head which curve rate as same as the channel cavity, and at two sides of the later part of the round head respectively has one incision, making the septum swing towards any one direction, using for to block the channel which flowing fluid needs not to flow into; the two cavities of the posterior end of the structure of double cavity fluid-air channel sheathe on two rigid connecting tubes respectively, between these two tubes has a welding pad piece, the posterior end of rigid connecting tubes again connect with two fluid-air channels adapters.

6. The endoscope system with a disposal sheath, according to the claim 1, the fluid-air channel may be a single cavity structure which came from two single cavity channels join in together one channel at nearing the jet channel site, and the channel shape may be cylinder or flat; or the structure of two single cavity fluid-air channels via heat pressure become flat channel and parallel with each other and form thin wall channels, and can be inflated and expanded into cylinders while injecting into fluid or air.

7. The endoscope system with a disposal sheath, according to the claim 1, characterized in that the three-way sealing cap is made of elastic medical material, its anterior end is fixed on the endoscope channel exit, its straight cavity is the way for inserting medical instrument and tight fixing the disposal channel; its side exit is a sucking channel adapter which connects the sucking channel, in the three-way sealing cap set three elastic sealing orifices, to elastically sealing the disposal channel at the guide tube's assistance.

8. The endoscope system with a disposal sheath, according to the claim 7, characterized in that the temporary guide tube can be sheathed in the three-way sealing cap, in advance, for sheathing the disposal channel in, which diameter is quite large; the guide tube consists of a tube body with a handle, guide tube core, a long orifice, and a orientation pin, the long orifice is symmetrically set in the tube body to allow the orientation pin crossed the guide tube core to slide forward and backward in it; the distance between the handle end face and posterior end of the long orifice is equal to the distance between the upper orifice of the three-way sealing cap and lower end face of the third elastic sealing orifice, the long orifice's length is more than the distance between the lower end of the guide tube and the first elastic sealing orifice of the three-way sealing cap; while the handle end face closes to the upper orifice of the three-way sealing cap, the orientation pin of tube core is pushed over the third elastic sealing orifice by the posterior end of the guide tube wall's long orifice, so that the tube core's lower end orientates to inferior to the second elastic sealing orifice; while the three-way sealing cap is installed on the endoscope, the tube core's lower end makes the posterior end of the disposal channel which is higher than the sucking channel orifice, being pressed to under the sucking channel orifice to prevent the sucking channel orifice from block; while pulling out the guide tube, although the guide tube's lower end is extracted out the first elastic sealing orifice, due to the space provided by the long orifice's length, the tube core's lower end cannot raise the disposal channel because the orientation pin of tube core is yet sited under the third elastic sealing orifice, and the disposal channel instantly is tight contracted and fixed, while all of the guide tube is pulled out, the disposal channel is closed up again and fixed by the second elastic sealing orifice, make the posterior end put in right position of the three-way sealing cap.

9. The endoscope system with a disposal sheath, according to the claim 1, characterized in that a V-shape electric resistance thread is on one piece of two pieces of heat fusion forceps, another piece has an insulation board, wherein one piece can move to all the channel orifice, and tight contact with electric resistance thread, the channel orifice is cut into different V-shapes according to different electric resistance thread's shapes of the forceps orifice.

10. The endoscope system with a disposal sheath, according to the claim 1, characterized in that the traction forceps can pull the disposal channel out the endoscope channel, one piece of forceps is a cylinder structure, the other piece is a half bucket size; while two pieces of the forceps orifice are closed, they can keep maximal contacting area with the channel wall, on forceps handle a teeth locking apparatus is set.
